(19) United States
(12) Patent Application Publication
(10) Pub. No.: US 2021/0368782 A1
(43) Pub. Date: Dec. 2, 2021

(54) NUCLEATED CELL PRESERVATION BY LYOPHILIZATION

(71) Applicant: CELLPHIRE INC., Rockville, MD (US)

(72) Inventors: Joshua Dee, Gilroy, CA (US); Anna Yu, Annandale, VA (US); Glen Michael Fitzpatrick, Gaithersburg, MD (US); Richard O. Cliff, Vienna, VA (US)

(73) Assignee: CELLPHIRE INC., Rockville, MD (US)

(21) Appl. No.: 17/401,474

(22) Filed: Aug. 13, 2021

Related U.S. Application Data
(62) Division of application No. 16/069,675, filed on Jul. 12, 2018.
(60) Provisional application No. 62/278,540, filed on Jan. 14, 2016.

(51) Int. Cl. A01N 1/02; C12N 5/078; A61L 31/00; A61L 31/08; A61L 31/16; C12N 5/0781; C12N 5/0783; C12N 5/0775

(52) U.S. Cl. CPC A01N 1/0284; A01N 1/02; C12N 5/0634; A61L 31/005; A61L 31/08; A61L 31/16; A61L 2300/64; C12N 5/0636; C12N 5/0663; A61L 2300/404; A61L 2300/408; A61L 2300/606; C12N 5/0635

(57) ABSTRACT

The invention provides freeze-dried nucleated cells, a method for preparing them, and methods of using them for in vitro assays and in vivo therapeutic treatments. The method for preparing the cells includes incubating cells in the presence of a cryoprotective sugar to load them with the sugar, then lyophilizing them without separating the cells from the cryoprotective sugar. In embodiments, the cells are also loaded with one or more bioactive agents.

### CROSS-REFERENCE TO RELATED APPLICATIONS

This application is a Divisional Application of U.S. patent application Ser. No. 16/069,675 filed on Jul. 12, 2018; which is a National Stage application under 35 U.S.C. § 371 of International Patent Application No.: PCT/US2017/012836 filed on Jan. 10, 2017, which claim the benefit of priority to U.S. Provisional Application No. 62/278,540, filed on Jan. 14, 2016. These applications are incorporated by reference herein in their entireties.

### BACKGROUND OF THE INVENTION

### FIELD OF THE INVENTION

The present invention relates to the fields of medicine, medical diagnostics, and cell-based technologies. Specifically, the invention relates to methods for making and using freeze-dried nucleated cells in medical treatments and diagnostics and as cell-based components in in vivo and in vitro systems incorporating cells for detection and sensing.

### SUMMARY OF THE INVENTION

The present invention provides a process for preparing freeze-dried (used interchangeably with “lyophilized”) nucleated cells. In general, the process includes contacting a population of nucleated cells with a cryoprotectant under conditions that allow the cryoprotectant to be internalized by the nucleated cells (referred to herein at times as “loading the cells”), contacting the “loaded” cells with an excipient or bulking agent, and optionally with proteins, including but not limited to cryoprecipitated proteins, to create a lyophilization mixture, and lyophilizing the mixture. It was unexpectedly found that the process of the invention provides a population of freeze-dried nucleated cells having a relatively high proportion of viable cells upon rehydration after lyophilization as compared to other processes for preparing lyophilized nucleated cells. The process thus provides a much needed advancement in the field of medicine and in particular the field of preservation of nucleated cells. While others in the art have developed protocols for freeze-drying of nucleated cells, those protocols have not met with widespread use due to their inability to provide medically useful levels of viable cells upon rehydration.

The present invention also provides a process for preparing rehydrated (used interchangeably with “reconstituted”) nucleated cells, where the process includes contacting the freeze-dried nucleated cells with an aqueous composition under conditions where the freeze-dried cells internalize at least the water of the composition to cause rehydration of the cells. The aqueous composition can be provided in the form of a liquid water composition, a water vapor composition, or a combination of the two. Preferably, the aqueous composition is present as a liquid composition.

The present invention further provides processes of using the freeze-dried and reconstituted freeze-dried cells of the invention. In one general embodiment of this aspect of the invention, the process is a process of medical treatment of a subject in need thereof. In general, the process includes administering to a subject the reconstituted freeze-dried nucleated cells of the invention in an amount that is adequate to treat a disease, disorder, or injury of the subject. Typically, the treatment is ameliorative or curative; however, in some embodiments it is prophylactic. The step of administering can be any action that results in contact of the reconstituted freeze-dried cells with the interior or exterior of the body of the subject. The process of medical treatment thus can be a process for internal administration or topical administration.

It is to be understood that the freeze-dried nucleated cells of the invention are stable over long periods of time not only at relatively cold temperatures (i.e., 4° C. or below) but at higher temperatures (e.g., about room temperature) as well. The invention thus provides for long-term preservation of nucleated cells. For example, the invention provides for preservation of cell lines without the need for expensive liquid nitrogen storage. The freeze-dried cells can be reconstituted at an appropriate time for use in vivo, such as for replacement of blood cells, including hemopoietic cells, such as bone marrow cells, including bone marrow stem cells. They can also be reconstituted or used directly for in vitro cell culture for diagnostic assays, such as cell-based detection assays. The in vitro uses are not particularly limited, and can be any use suitable for the type of nucleated cell that is freeze-dried. For example, the freeze-dried cells can be used as controls in functional assays requiring living cells, such as white cell—LPS interaction assays, controls for FACS assays where fixed cell membranes are not desirable, and other assays where metabolic interactions between cells and compounds are needed, such as apoptotic assays for toxicity. As yet another non-limiting example, stabilized cancerous cells can be used as a standard platform for testing anti-cancer or other anti-proliferative drugs. Yet again, the cells can be used in immunoassays as a source of stabilized antibody-presenting cells.

One notable aspect of the invention is the ability to create freeze-dried nucleated cells that contain (i.e., are loaded with) one or more bioactive agents. That is, the process of loading the cells can include loading the cells with a bioactive agent prior to freeze-drying, which produces a cell that, when rehydrated, can deliver the bioactive agent to a subject in vivo or to a cell culture or assay in vitro. The bioactive agent can be any substance that has a chemical, biochemical, or physiological effect on the cell itself or other cells present in the same environment as the rehydrated nucleated cell. In embodiments, the bioactive agent is a therapeutic substance for delivery in vivo to treat or prevent a disease or disorder. As those of skill in the art understand, the act of prevention does not require 100% efficacy. Non-limiting examples of bioactive agents are drugs, such as antibiotics, antifungal, antiviral, and antimitotic agents.

### BRIEF DESCRIPTION OF THE DRAWING

The accompanying drawing, which is incorporated in and constitutes a part of this specification, illustrates embodiments of the invention, and together with the written description, serves to explain certain principles and advantages of the invention.

FIG. 1 is a table showing the proportion of viable nucleated cells achieved using various embodiments of the process of the invention.

### DETAILED DESCRIPTION OF VARIOUS EMBODIMENTS OF THE INVENTION

Reference will now be made in detail to various exemplary embodiments of the invention. It is to be understood that the following detailed description is provided to assist the reader in understanding certain features and embodiments of the invention, and that the following detailed description is not to understood as limiting the invention to the particular details specifically discussed.

Before embodiments of the present invention are described in detail, it is to be understood that the terminology used herein is for the purpose of describing particular embodiments only, and is not intended to be limiting. Further, where a range of values is provided, it is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which the term belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. The present disclosure is controlling to the extent it conflicts with any incorporated publication.

As used herein and in the appended claims, the singular forms “a”, “an”, and “the” include plural referents unless the context clearly dictates otherwise. Thus, for example, reference to “a cell” includes a plurality of such cells and reference to “the sample” includes reference to one or more samples and equivalents thereof known to those skilled in the art, and so forth. Furthermore, the use of terms that can be described using equivalent terms include the use of those equivalent terms. Thus, for example, the use of the term “subject” is to be understood to include the terms “patient”, “animal”, “human”, and other terms used in the art to indicate one who is subject to a medical treatment. As another example, the use of the term “neoplastic” is to be understood to include the terms “tumor”, “cancer”, “aberrant growth”, and other terms used in the art to indicate cells that are replicating, proliferating, or remaining alive in an abnormal way.

In a first aspect, the invention is directed to a process for preparing freeze-dried nucleated cells. In general, the process includes loading nucleated cells with a cryoprotectant, contacting the loaded cells with 1) an excipient or bulking agent and 2) one or more proteins, to create a lyophilization mixture, and lyophilizing the mixture. According to the process, the cells are not removed from the solution used for loading of the cells before the cells are contacted with the excipient/bulking agent and proteins. As such, the process does not include a separation step, such as a centrifugation step, between loading of the cells and lyophilization of the cells. Preferably, the proteins comprise cryoprecipitated proteins.

Nucleated cells according to the invention are all cells that have a nucleus. The invention thus encompasses all nucleated cells of eukaryotic organisms. The cells discussed and detailed herein are cells of the blood system; however, it is to be understood that the invention is not limited to such cells. Exemplary blood cells include: white blood cells (leukocytes), such as neutrophils, eosinophils, basophils, lymphocytes, and monocytes; and bone marrow cells, such as hematopoietic stem cells. Among the lymphocytes, all of the various B-cells and T-cells are encompassed by the invention. Importantly, it is to be recognized that the invention relates in embodiments to a single type of cell, such as a bone marrow cell. Yet in other embodiments, the invention relates to a mixture of two or more types of cells. In non-limiting exemplary embodiments discussed herein in detail, the invention relates to a mixture of the various different types of nucleated cells found in blood. In other non-limiting examples, the invention relates to umbilical cord blood. Yet other non-limiting examples relate to bone marrow cells or other pluripotent or totipotent cells, such as stem cells, which can be used therapeutically by themselves or to augment cell types of interest through therapeutic delivery of the cells. Mention can also be made of pancreatic cells, which can be used in treatment of diabetes. As should be evident, in some embodiments, the nucleated cells are all of the same type. For example, nucleated cells according to embodiments of the invention may be all or substantially all B-cells, all or substantially all T-cells, all or substantially all monocytes, all or substantially all lymphocytes (in any proportion of B-cells and T-cells), etc.

The process of preparing lyophilized nucleated cells optionally includes obtaining or preparing the nucleated cells for loading and lyophilization. Obtaining or preparing the cells can be any action that results in providing purified or isolated nucleated cells for subsequent use in the process. For example, cells may be isolated by any of several standard techniques, including, but not limited to: centrifugation, tissue culture, affinity column binding, FACS, filtration, or other techniques standard in the art. Within the context of blood cells, many suitable protocols are known in the art for separating white blood cells from red blood cells, platelets, and plasma, and any such protocols can be used. Preferably, a protocol that involves centrifugation of whole blood to separate the various components from each other is used. For example, the commercially available BD brand CPT blood draw tube can be used for centrifugation-driven separation of white blood cells from other blood components. In general, for centrifugation-driven separation of white blood cells, conditions of centrifugation at room temperature for 25 minutes at 1,700×g, or equivalent conditions, are suitable. As is known in the art, cells separated from other cells or biological material can be washed one or more times to enhance purity.

The process includes loading nucleated cells with a cryoprotectant. Loading of the cells results from contacting the nucleated cells with a cryoprotectant for an amount of time and under appropriate conditions whereby the cryoprotectant is taken up by the cells. Contacting thus can be exposing the cells to the cryoprotectant by combining, mixing, etc. the two in an aqueous environment. Loading a cryoprotectant into the nucleated cells is believed to protect the cells from lysis and to promote retention of viability during lyophilization and rehydration. The cryoprotectant can be any of the known substances suitable for protection during lyophilization of cells, such as platelets. Exemplary embodiments include the use of a sugar, such as trehalose. While not being bound by any particular mode of operation, entry of the cryoprotectant into the cells is believed to occur through a process of thermal endocytosis. In general, for loading of trehalose into the cells, the cells are exposed to trehalose from one to four hours at a temperature of between 25° C. and 40° C. In preferred embodiments, the cells are incubated in the presence of trehalose for 2 hours at 37° C. Optionally, the combination of cells and cryoprotectant can be gently agitated, such as by inversion of the incubation chamber, periodically, such as every 15-60 minutes, preferably every 30 minutes.

The loading composition is an aqueous solution of at least the cells and the cryoprotectant. In exemplary embodiments, trehalose is used as the cryoprotectant, and it is present in an amount of from 30 mM to 250 mM, such as from 50 mM to 150 mM or 75 mM to 125 mM. Preferably, trehalose is present at a concentration of about 100 mM. While the optimal amount of cryoprotectant can vary based on the type of cell and the identity of the cryoprotectant, it has been found that, when trehalose is used, no advantage is seen when the trehalose concentration exceeds 500 mM. Further, when blood cells are being lyophilized, there does not appear to be any advantage to using trehalose in a concentration greater than 150 mM.

The loading composition can comprise optional components, which can improve the ability to prepare freeze-dried cells that are viable upon rehydration. One optional component of the loading composition is ethanol, which can be present in an amount of 0.1% to 2% (v/v), such as about 1%. Additionally or alternatively, fibrinogen can be included in an amount of 0.1% to 2% (w/v), such as 0.1% to 1.5%, either by itself or as part of a cryoprotein composition. Where fibrinogen is used, it is preferably present at about 1.5% in the loading composition. The loading composition is preferably a buffered aqueous solution that includes at least a buffer, a salt, and a sugar, which in embodiments where a sugar is used as a cryoprotective agent, is a different sugar than the cryoprotective agent. In general, the identities of components are not critical as long as they are biologically tolerable at the concentrations used. Thus, for example, the buffer can be HEPES, bicarbonate, or another buffer or combination of buffers that is suitable for use in maintaining pH at a relatively neutral range, such as pH 6.2-7.8. In addition, the salt can be any biologically tolerable salt or combination of salts, where each salt or the combination is in the range of from about 3 mM to 150 mM, such as about 5 mM to 100 mM, about 5 mM to about 75 mM, or about 50 mM. Likewise, the sugar can be present in an amount ranging from about 2 mM to about 50 mM, such as from about 2 mM to about 20 mM, about 3 mM to about 10 mm, or about 5 mM. In exemplary embodiments, the loading buffer comprises: 9.5 mM HEPES, 75 mM NaCl, 4.8 mM KCl, 12 mM NaHCO₃, and 5 mM glucose (dextrose). In general, the composition should be isotonic to the cells to avoid shrinking, swelling, or other deleterious effects on the cells.

As mentioned above, the freeze-dried cells and rehydrated cells produced from them can include one or more bioactive agents. When present, the bioactive agents are introduced into the cells prior to lyophilization, typically at the time of loading the cells with cryoprotectant. The bioactive agents can be provided for any purpose, but in general do not contribute to cryoprotection or other aspects of production of the freeze-dried cells per se. One class of bioactive agents contemplated by the invention are therapeutic substances, such as those generally referred to as drugs. These substances are typically released by the cells upon rehydration and use in vivo and in vitro. The identity of each bioactive agent is not critical, although it is recognized that only agents that are of a sufficiently small size to be taken up by the cells during the loading process will be suitable for use in the invention. Among the numerous bioactive agents useful in the invention, non-limiting examples include antimicrobial agents (e.g., antibiotics, antivirals, antifungals), growth factors, anti-apoptotic agents, chemotherapeutic agents, antimitotic agents, hormones, and anti-toxins. While not being limited to any particular mode of action, it is presumed that the bioactive agents are taken up via the same process as the cryoprotectant. The skilled artisan will recognize that co-loading of bioactive agents with cryoprotectant is not a required feature of the invention, but instead provides additional advantages to the freeze-dried cells and rehydrated freeze-dried cells in embodiments.

Furthermore, the freeze-dried nucleated cells and rehydrated cells produced from them can include one or more labeling agents or other markers for cells or biochemical activity. As with the bioactive agents discussed above, the labeling agents/markers are introduced into the cells prior to lyophilization, such as at the time of loading the cells with cryoprotectant. Non-limiting examples of labeling agents/markers are fluorescein, bodipy, and ICG.

In addition to loading the nucleated cells with a cryoprotectant, the process of making freeze-dried nucleated cells includes contacting the loaded cells with 1) an excipient or bulking agent to create a lyophilization mixture. It can, in embodiments, also include contacting the loaded cells with one or more proteins to create a lyophilization mixture. The excipient/bulking agent is added such that its final concentration in the lyophilization mixture is between 0.1% and 10% (w/v), such as between 1% and 10%, between 2.5% and 7.5%, or about 5%-6%. Excipients/bulking agents useful in the lyophilization mixture are excipients/bulking agents known in the art, and include but are not limited to, polysucrose 400 and Ficoll® 400 (both are copolymers of sucrose and epichlorohydrin), polyvinylpyrrolidone 40, maltose, and albumin. Preferably, polysucrose 400 or Ficoll® 400 is used at a final concentration of 6%. When included, the proteins used can be any suitable protein. In some embodiments, the proteins comprise cryoprecipitated proteins from blood. Alternatively or additionally, albumin, such as BSA or HSA is present.

According to the invention, cryoprecipitated proteins (cryoproteins) are plasma proteins that are found as an insoluble fraction of the plasma after frozen plasma has been thawed at 1oC. to 6oC. The material contains factor VIII, fibrinogen, fibronectin, factor XIII, and VonWillebrand factor (vWf). Cryoprecipitated proteins are optional components of the lyophilization mixture. When present, they preferably comprise 0.1% to 50% (v/v) of the final volume of the mixture. To achieve that concentration, a standard cryoprecipitate solution for addition to the lyophilization mixture can be made from 50 ml of plasma, which is, in embodiments, fibrinogen-depleted. The plasma is centrifuged to pellet the cryoproteins, which are then resuspended in 5 ml (resulting in a 10× concentrated solution as compared to plasma). The 10× stock is added to the lyophilization mixture at a suitable ratio to achieve a desired concentration of cryoproteins. For example, the stock solution can be added to the lyophilization mixture at a 1:25 ratio (4% v/v). As such, 40% of the cryoproteins one would expect from an equivalent volume of plasma is added to the lyophilization mixture.

During or shortly after addition of the substances of the lyophilization mixture to the loading composition, the cell concentration should be adjusted to within 10% of the desired final concentration. Additional dilution may be performed by addition of loading buffer and excipients in proportional amounts, should counts be higher than desired. Loaded cells in complete lyophilization buffer are then dispensed into serum vials or other appropriate lyophilization vessels standard in the art. Preferably, the cell mixture is introduced into the lyophilization vessel such that the volume of cell mixture is one-fifth of the volume of the lyophilization vessel. For example, 1 ml of cells is added to a 5 ml lyophilization tube, 20 ml of cells are added to a 100 ml lyophilization bottle, etc. The lyophilization vessels then can be loosely stoppered, and placed into a lyophilization chamber.

The process of making freeze-dried nucleated cells further includes lyophilizing the lyophilization mixture. Samples can be lyophilized according to the following parameters. Freezing is performed between −40° C. and −90° C. for one to six hours, after which primary drying is carried out below the glass transition temperature (Tg) point of the material. Typically, this requires drying at a temperature between −30° C. and −50° C. for about 5 to 15 hours, preferably about 10 hours. Secondary drying is then carried out above the Tg, such as between 10° C. and 40° C., preferably between 25° C. and 30° C., for about 3 to 10 hours, preferably about 5 hours. The cells are then held under vacuum at between 20° C. and 30° C. until removed from the lyophilizer. Table 1 shows exemplary lyophilization cycle conditions.

TABLE 1
Exemplary Lyophilization Conditions

| Temperature (.° C.) | Time (Minutes) | Vacuum (milliTorr) |
|---|---|---|
| −50 | 70 ramp | |
| −50 | 180 hold | |
| −30 | 60 ramp | <200 |
| −30 | 540 hold | <200 |
| 30 | 60 ramp | <200 |
| 30 | 240 hold | <200 |
| 25 | Hold until removed | <200 |

Once lyophilization is complete, the vessels/containers/vials are stoppered under a vacuum of less than 200 mTorr and then removed from the lyophilizer. Optionally, the stoppered vessels can be heat-treated at a temperature between 60° C. and 85° C. for about 12 to 36 hours. Where post-lyophilization heat treatment is used, it is preferred that treatment is at 80° C. for 15 to 24 hours.

The present invention also provides a process for preparing rehydrated nucleated cells. In brief, the process includes contacting the freeze-dried nucleated cells of the invention with an aqueous composition under conditions where the freeze-dried cells internalize at least the water of the composition to cause rehydration of the cells. The step of contacting can be any action that results in the water coming into physical contact with the freeze-dried cells and being taken into the cells to rehydrate them. In preferred embodiments, an aqueous composition is added to the vessel containing the freeze-dried cells to effect rehydration. The cells are then allowed to rehydrate. If desired, gentle agitation of the vessel can be performed to separate the dried cells and accelerate rehydration of the cells. The aqueous composition can include, in addition to water, any number of additional components, such as those known in the art as suitable for maintenance of nucleated cells in a viable state. Such components, and such compositions, are well known and widely used in the art, and thus need not be listed here. For example, freeze-dried nucleated cell samples can be rehydrated with water, or a water/buffer/plasma mixture. In some embodiments, the cells are rehydrated in a volume of water that is equal to the volume of the lyophilization mixture added to the vial before lyophilization.

The freeze-dried nucleated cells of the invention are useful in a wide range of applications in the medical field. Among the many uses, mention can be made of use in medical treatments. Those of skill in the art can envision numerous medical applications for freeze-dried nucleated cells, and all such applications are encompassed by the present invention. While uses for the freeze-dried nucleated cells of the invention are discussed in detail herein with respect to blood cells, the various uses for other types of cells, including cells of other organs and systems of the body, are also contemplated.

One aspect of the invention relates to a process of medical treatment of a subject in need thereof. The process includes administering to a subject in need an appropriate amount of the freeze-dried nucleated cells of the invention in an amount that is adequate to treat a disease, disorder, or injury in a subject suffering from, suspected of suffering from, or at risk of developing the disease, disorder, or injury. Preferably, the freeze-dried nucleated cells are rehydrated prior to administration to the subject. In certain embodiments, the process of treatment treats a disease or disorder that results from an infection or trauma to the subject. In other embodiments, the process treats a disease or disorder that results from genetic or environmental factors, including, but not limited to, neoplasias. In yet other embodiments, the process treats side-effects of other treatments applied to a subject. For example, the process can be a process of treating a patient undergoing chemotherapy, who has a low white blood cell count due to the chemotherapy. Such a treatment can be, for example, bone marrow replacement for ablative immune therapy in treatment of leukemia. Other treatments include stem cell transplant and liver cell transplant. Of course, the process can conversely be thought of as a process of treating a disease, disorder, or injury rather than a process of treating the subject. According to the invention, treatment of one is tantamount to treatment of the other.

In one aspect of the methods of treating a subject, rehydrated freeze-dried nucleated cells are seeded onto a scaffold and preferably allowed to adhere to and grow on the scaffold prior to administration to the subject. For example, the rehydrated cells can be seeded and grown on a stent prior to implantation of the stent into a patient. As another non-limiting example, rehydrated cells are seeded and preferably grown on a wound dressing matrix, such as one known in the art for topical treatment of wounds. In some embodiments relating to administration in conjunction with a scaffold, the rehydrated nucleated cells are loaded with a bioactive agent that enhances the therapeutic effectiveness of the cells and scaffold. For example, where cells are seeded onto a wound dressing matrix, preferably the cells are loaded with one or more antibiotics, which, when released by the cells, decrease the likelihood of developing or even completely prevent bacterial infections during the wound healing process. Likewise, where cells are seeded onto a stent for repair of a blood vessel, preferably the cells are loaded with one or more agents that deter cell proliferation to reduce the chance of restenosis and occlusion of the vessel being treated. In embodiments relating to scaffolds, administration of the scaffold-cell combination (which can be considered a medical device) can include surgical implantation of the device into the subject.

Those of skill in the medical and veterinary arts are well aware of the various types of scaffolds available for treatment of patients, and the techniques used to seed such scaffolds with cells. Such scaffolds include, but are not limited to, reconstructive matrices and scaffolds for regenerative therapy (e.g., for cartilaginous tissues). The skilled artisan may use any suitable combination of scaffolds and cells to achieve the desired results.

The step of administering can be any action that results in contact of the freeze-dried cells with the interior or exterior of the body of the subject. Administering thus can be as simple as pouring, sprinkling, or spraying the freeze-dried cells or rehydrated freeze-dried cells onto the surface of a subject's body. Administering can also be by way of oral administration of a capsule, pill, etc. Likewise, administration can be by way of capsules, pills, powders, and the like to mucosal surfaces. It is to be noted that administration includes direct delivery of the cells to a site of interest, systemic delivery of the cells to the entire body of the subject, and localized delivery of the cells to a particular site of interest. In embodiments, administering comprises injection or infusion of rehydrated freeze-dried nucleated cells into the blood system of the subject being treated.

The number of freeze-dried cells or rehydrated freeze-dried cells to be delivered will vary depending on the disease, disorder, or injury being treated, the size of the subject, and other factors. The appropriate number can be determined by the skilled artisan without undue or excessive experimentation.

The process of treatment according to the invention is useful for treating all manner of subjects. Non-limiting examples of subjects for treatment include humans, companion animals (e.g., dogs, cats, rodents), agricultural animals (e.g., horses, cows, sheep, goats), and wild animals (e.g., those maintained in zoos, endangered species). The invention thus has use in both medical and veterinary applications.

As discussed above, the present invention provides processes of using the rehydrated freeze-dried cells of the invention. As with use of the freeze-dried nucleated cells, this aspect of the invention includes a process of medical or veterinary treatment of a subject in need thereof The process includes administering to a subject in need thereof an appropriate amount of the reconstituted nucleated cells according to the invention. According to this aspect of the invention, administration relates to delivery of a liquid or liquid-like (e.g., gel, salve) composition to the subject. In accordance with the discussion above, the number of rehydrated cells to be delivered will vary depending on the disease, disorder, or injury being treated, the size of the subject, and other factors. The reconstituted freeze-dried nucleated cells find similar medical uses as the freeze-dried nucleated cells themselves.

One aspect of the invention relates to populations of freeze-dried nucleated cells and populations of rehydrated freeze-dried nucleated cells. Populations according to the invention have a relatively high percentage or proportion of viable cells, as compared to prior art attempts by others to create such populations. The populations show cell viability after reconstitution at levels comparable to recovery rates achieved by DMSO cryopreservation. Yet the cells of the present invention do not have the drawbacks associated with DMSO cryopreservation. It has surprisingly been found that cell viability levels in populations according to the present invention can reach or exceed 20%. Depending on the particular cells and parameters used, viability levels can reach or exceed 7%, 10%, 15%, 20%, 25%, 30%, 35%, 40%, 45%, 50%, 55%, 60%, 65%, 70%, 75%, 80%, 85%, 90%, 95%, or more. Populations according to the invention are typically in vitro compositions, such as a population of cells contained in a vessel, container, vial, syringe, etc., which are maintained or grown until used in in vivo or in vitro applications. The compositions typically contain, in addition to the cells, an aqueous environment that is suitable for maintaining the cells in a viable state until they are used for the various purposes that cell compositions are used, including those discussed herein.

Kits are useful in the present invention for packaging and delivering the freeze-dried nucleated cells and cell populations. The kits include the cells and/or populations in one or more containers. While a container (e.g., lyophilization vessel, serum vial) can be considered as a form of a kit, typically, a kit of the invention comprises multiple containers containing cells and/or populations, packaged in combination. Kits can be made of any suitable material, including but not limited to cardboard, plastic, glass, and metal. In certain embodiments, kits contain one or more containers of a population of freeze-dried or reconstituted nucleated cells, where the cells are provided in each container in an amount sufficient to practice a method of treatment according to the invention. Additional optional components of the kits include water or an aqueous solution for rehydration/resuspension of the freeze-dried cells, vials or other containers for transfer or growth of the rehydrated cells, and/or reagents and other materials needed to administer reconstituted cells to a subject or to practice an in vitro assay using the cells.

To recapitulate, the present invention is directed, in certain aspects, to a population of freeze-dried nucleated cells, wherein the population, when rehydrated, has a viability level of at least 20%, such as at least 25%, or at least 35%. In embodiments, the population of cells includes at least some cells that comprise a bioactive agent, such as an antibacterial agent, an antiviral agent, or an antifungal agent. In embodiments, the cells are mammalian cells, such as human cells. In embodiments, the cells are blood cells, including, but not limited to B-cells, T-cells, and stem cells, such as bone marrow stem cells. In other aspects, the invention is directed to a method for preparing freeze-dried nucleated cells. In embodiments, the method comprises loading nucleated cells with a cryoprotectant in an aqueous environment; contacting the loaded cells with an excipient or bulking agent to create a lyophilization mixture; and lyophilizing the mixture, wherein the method does not include a separation step between loading of the cells and lyophilizing the cells. In embodiments, the method can further comprise, prior to lyophilizing the mixture, contacting the loaded cells with one or more proteins, such as cryoprecipitated proteins. In exemplary embodiments, the method includes the use of a cryoprotectant that comprises trehalose at a concentration of about 100 mM. In preferred embodiments, the method includes loading the cells with trehalose as the cryoprotectant for 2 hours at 37° C. In exemplary embodiments, the excipient or bulking agent is polysucrose 400, which is present in the aqueous environment at a concentration of 6% (w/v). In addition, in embodiments the method can further comprise loading the nucleated cells with one or more bioactive agents, such as an antibacterial agent, an antiviral agent, or an antifungal agent. For some cell types, a post-lyophilization heating step can be beneficial. For example, after lyophilization, the cells can be heated at about 80oC. for 15-24 hours. Various non-limiting examples of cells that are suitable for use in the present method include mammalian cells, such as human cells or canine cells. Other non-limiting types of cells include blood cells, such as B-cells, T-cells, or stem cells, including, but not limited to bone marrow stem cells. The invention further encompasses freeze-dried nucleated cells made by the method of the invention as well as rehydrated freeze-dried nucleated cell produced by the method of the invention, which further comprises rehydrating the lyophilized cells. Yet again, the invention encompasses a medical device comprising rehydrated nucleated cells produced by a method of the invention. The medical device can, in embodiments, comprise a scaffold onto which the rehydrated nucleated cells are adhered. In embodiments, rehydrated nucleated cells of the invention comprise one or more bioactive agents. In another aspect, the invention encompasses a method of treating a subject suffering from a disease, disorder, or injury, where the method comprises administering to the subject a population of rehydrated freeze-dried nucleated cells, wherein the population has a viability level of at least 20%, and wherein the population of cells is administered in an amount sufficient to treat the disease, disorder, or injury. In embodiments, the method can be a method of treating a disease or disorder involving the blood system, and the step of administering comprises administering rehydrated hemopoietic cells, such as bone marrow stem cells.

### EXAMPLES

The invention will be further explained by the following Examples, which are intended to be purely exemplary of the invention, and should not be considered as limiting the invention in any way. It is to be understood that the following Examples disclose specific materials and reagents from commercial vendors, but that equivalent materials and reagents from other vendors can be substituted, unless otherwise indicated.

### Example 1

Preparation of Freeze-Dried Nucleated Cells

Whole blood was drawn from a human donor directly into Becton Dickinson (BD) CPT cell separation tubes and into lithium heparin tubes. Heparinized blood was transferred to CPT tubes in order to have similar sized tubes for centrifugation. All CPT tubes were clearly labeled as “heparinized” or “non-heparinized” to indicate the type of blood they contained. CPT tubes were centrifuged as per the BD instructional insert that shipped with the CPT tubes as follows. Cells were centrifuged for 25 minutes at 1700×g at room temperature. Plasma was removed above the buffy coat and the cell fraction of plasma above the separator was collected. Cell count was assessed using a Beckman Coulter Act-10. Samples were brought up to 15 ml with PBS-EGTA, capped, and inverted 5 times to mix. Samples were then centrifuged for 15 minutes at 300×g. Supernatant was aspirated without disturbing the cell pellet. Cells were resuspended in a minimal volume (10 ml) of PBS-EGTA and the cell count was assessed. A second centrifugation of the removed supernatant was performed to increase the yield of cells. Supernatant was centrifuged for 20 minutes at 400×g. Resuspended cells were combined with the cells of the first wash step. The cells were separated into four aliquots to allow for four different preparation protocols. Two of the aliquots were processed according to “Preparation A”, below, and two of the aliquots were processed according to “Preparation B”, below. In sum, two aliquots were subjected to the Preparation A protocol: one aliquot with heparinized cells and one aliquot with non-heparinized cells; and two aliquots were subjected to the Preparation B protocol: one aliquot with heparinized cells and one aliquot with non-heparinized cells.

Preparation A Protocol:

Cells were centrifuged for 10 minutes at 300×g. The supernatant was aspirated and the cell pellet was resuspended in 2 ml of “Prep A Loading Buffer” (below). The final volume was adjusted to reach a targeted cell concentration count of 1.25-1.50×10³/μl in Prep A Loading Buffer.

Prep A Loading Buffer:

9.5 mM HEPES

75 mM NaCl

4.8 mM KCl

5 mM glucose (dextrose)

12 mM NaHCO₃

100 mM α,α-Trehalose

1% EtOH (v/v)

Sample tubes were sealed and incubated at 37° C. for 2 hours with gentle agitation every 30 minutes. After incubation, polysucrose 400 (stock 30% w/v solution) was added to reach a final polysucrose concentration of 6% and a final cell concentration of 1.0×10³/μl. Cells (1 ml) were dispensed into 5 ml lyophilization vials. Using the lyophilization cycle of Table 1, above, samples were freeze-dried using a VirTis advantage lyophilizer using a Wizard 2.0 control board and software version 5.1.

### Preparation B Protocol:

Cells were centrifuged for 10 minutes at 300×g. The supernatant was aspirated and the cell pellet was resuspended in 2 ml of Prep B Loading Buffer.

Prep B Loading Buffer

9.5 mM HEPES

75 mM NaCl

4.8 mM KCl

5 mM glucose (dextrose)

12 mM NaHCO₃

200 mM α,α-Trehalose

1% EtOH (v/v)

The final volume of the prep was established to determine the target cell concentration of 1.30-1.55×10³/μl in Prep B Loading Buffer and then 1.25 mg/ml of fibrinogen from a concentrated stock solution (40-50 mg/ml) was added. Sample tubes were sealed and incubated at 37° C. for 2 hours with gentle agitation every 30 minutes. After incubation, polysucrose 400 (stock 30% w/v solution) was added to reach a final polysucrose concentration of 6%. Then, an additional 1/25 volume of cryoprotein was added for a final cell concentration of 1.0×10³/μl. Cells were dispensed (1 ml each) into 5 ml lyophilization vials. Using the lyophilization cycle shown in Table 1, above, samples were freeze-dried.

The vials from Prep A and Prep B were each divided into two groups, one of which was further treated by incubation at 80° C. for 15 hours.

According to the procedure, a total of eight different conditions resulted: Prep A—heparin; Prep A—non-heparin; Prep B—heparin; Prep B—non-heparin; Prep A—heparin+heat treatment; Prep A—non-heparin+heat treatment; Prep B—heparin+heat treatment; Prep B—non-heparin+heat treatment.

Samples were rehydrated with 1 ml of water and allowed 5-10 minutes for full rehydration. Cell viability was determined using a Trypan Blue exclusion test according to Strober, W. (“Trypan Blue Exclusion Test of Cell Viability”, Current Protocols in Immunology, 1997, A.3B.1-A.3B.2). For Trypan Blue analysis, 5 μl of Trypan Blue was added to 45 μl of undiluted rehydrated sample and mixed. A neat sample of 10 μl was added into the chamber of a hemocytometer and allowed to settle for 2-3 minutes. Under 450×magnification, counts were made of two populations: those that had excluded Trypan Blue (and had clear cytoplasm), and those that did not (and had blue cytoplasm). That is, clear cells were viable, whereas blue cells were dead. Cells were counted in five of the hemocytometer's 1/25 mm squares.

Hemocytometer calculation: the hemocytometer volume for the region counted is 0.1 mm deep, and covered five 1/25 square mm areas, or 5×0.04 mm^{2,} 0.1×(5×0.04)=0.02 cubic mm, or 0.02 μl of volume in which cells were counted. To determine cell count per ml, the number of cells counted was multiplied by 50,000 (1000 μl/0.02 μl) then divided by 0.9 to account for the volume of trypan blue added.

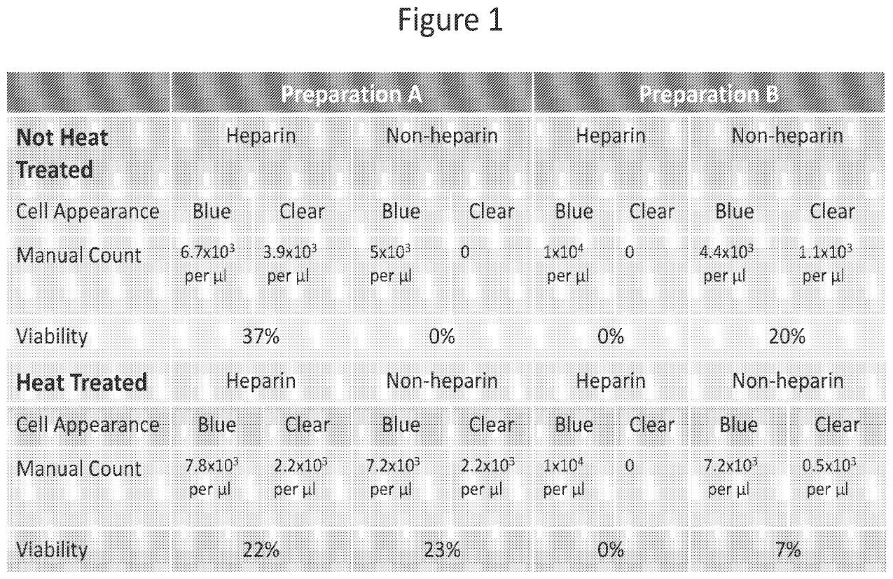

The results are shown in tabular form in FIG. 1. As seen in FIG. 1, multiple protocols falling within the general teachings of the present document were successful at preparing rehydrated freeze-dried nucleated cells (and thus freeze-dried nucleated cells). The data show that heparin treatment was important for the viability of cells prepared using the Preparation A protocol and not subjected to a post-lyophilization heat treatment step, whereas heparin treatment had no viability-enhancing effect on cells prepared using the Preparation A protocol and subjected to a post-lyophilization heat treatment step, or on cells prepared using the Preparation B protocol. Further, the data show that while in some cases heat treatment can improve viability (e.g., for cells not treated with heparin), it is not a necessary protocol step to achieve adequately high viability (e.g., heparin treated cells prepared using the Preparation A protocol). The data further show that the two protocols used can result in adequately high viability (e.g., Prep A—heparin; Prep B—non-heparin; Prep A—heparin+heat treatment; Prep A—non-heparin+heat treatment).

It will be apparent to those skilled in the art that various modifications and variations can be made in the practice of the present invention without departing from the scope or spirit of the invention. Other embodiments of the invention will be apparent to those skilled in the art from consideration of the specification and practice of the invention.

What is claimed is:

1. A method for preparing freeze-dried nucleated cells, said method comprising:
loading nucleated cells with a cryoprotectant in an aqueous environment, wherein the cryoprotectant is trehalose at a concentration between 30 mM and 250 mM, and wherein the loading comprises incubating the nucleated cells at a temperature of between 25° C. and 40° C. for 1 to 4 hours to form loaded cells;
contacting the loaded cells with an excipient or bulking agent comprising polysucrose to create a lyophilization mixture, wherein the excipient or bulking agent is present in the lyophilization mixture at a concentration of 1% to 10% (w/v);
lyophilizing the lyophilization mixture to form a lyophilized mixture; and
heating the lyophilized mixture at a temperature of between 60° C. and 85° C. for 12 to 36 hours, wherein the nucleated cells are blood cells.

2. The method of claim 1, wherein the blood cells comprise lymphocytes.

3. The method of claim 1, wherein the blood cells comprise B-cells and T-cells.

4. The method of claim 1, further comprising, prior to lyophilizing the mixture, contacting the loaded nucleated cells with one or more proteins, wherein the one or more proteins comprise cryoprecipitated proteins and/or albumin, wherein the one or more proteins is at a concentration of between 0.1% to 10% (w/v).

5. The method of claims 1, wherein the cryoprotectant comprises trehalose at a concentration of between 50 mM and 150 mM.

6. The method of claims 5, wherein the cryoprotectant comprises trehalose at a concentration of between 75 mM and 125 mM.

7. The method of claims 1, wherein the polysucrose is present in the lyophilization mixture at a concentration of 2.5% to 7.5% (w/v).

8. The method of claim 1, wherein the polysucrose is polysucrose 400.

9. The method of claim 1, wherein the aqueous environment further comprises a buffer and a salt at a concentration of 5 mM to 75 mM, wherein the pH of the aqueous environment is between 6.2 and 7.8, and wherein the aqueous environment is without dimethyl sulfoxide.

10. The method of claim 9, wherein the aqueous medium further comprises a sugar that is a different sugar than the cryoprotectant, wherein the sugar is present at a concentration of between 2 mM and 50 mM.

11. The method of claim 1, wherein the aqueous medium further comprises a sugar that is a different sugar than the cryoprotectant, wherein the sugar is present at a concentration of between 2 mM and 50 mM.

12. The method of claim 1, further comprising loading the nucleated cells with one or more bioactive agents.

13. The method of claim 12, wherein the bioactive agent is an antibacterial agent, an antiviral agent, or an antifungal agent.

14. The method of claim 1, wherein the heating the lyophilized cells is at 60° C. to 85° C. for 15-24 hours.

15. The method of claims 1, wherein the aqueous environment further comprises ethanol at a concertation between 0.1 and 2.0% (v/v).

16. The method of claims 15, wherein the aqueous environment further comprises a buffer and a salt at a concentration of 5 mM to 75 mM, and wherein the pH of the aqueous environment is between 6.2 and 7.8.

17. The method of claim 16, wherein the aqueous medium further comprises a sugar that is a different sugar than the cryoprotectant, wherein the sugar is present at a concentration of between 2 mM and 50 mM.

18. The method of claim 1, wherein the blood cells are human blood cells.

19. The method of claims 1, wherein the aqueous environment further comprises fibrinogen at a concertation between 0.1 and 2.0% (w/v).

20. The method of claims 1, wherein the blood cells are collected from heparinized blood.
